(19) United States
(12) Patent Application Publication
(10) Pub. No.: US 2025/0123633 A1
(43) Pub. Date: Apr. 17, 2025

(54) INFORMATION PROCESSING APPARATUS, INFORMATION PROCESSING METHOD, AND PROGRAM

(71) Applicant: Sony Group Corporation, Tokyo (JP)

(72) Inventors: Fumihiko IIDA, Tokyo (JP); Itaru SHIMIZU, Tokyo (JP)

(73) Assignee: Sony Group Corporation, Tokyo (JP)

(21) Appl. No.: 18/834,656

(22) PCT Filed: Jan. 26, 2023
(86) PCT No.: PCT/JP2023/002384

§ 371 Date: Jul. 31, 2024

(30) Foreign Application Priority Data
Feb. 10, 2022 (JP) 2022-019645

(51) Int. Cl. G05D 1/644; A61B 5/0205; A61B 5/16

(52) U.S. Cl. CPC G05D 1/645; A61B 5/0205; A61B 5/165

(57) ABSTRACT

The present disclosure relates to an information processing apparatus, an information processing method, and a program that can implement a robot system that is accepted by society.

An action planner plans an action of a mobile body on the basis of an acceptability level to the mobile body set for each of one or a plurality of users and an action parameter that is set on the basis of an action purpose of the mobile body and determines an action guideline of the mobile body. The technology of the present disclosure can be applied to a robot system including a transfer robot, for example.

### TECHNICAL FIELD

The present disclosure relates to an information processing apparatus, an information processing method, and a program, and particularly relates to an information processing apparatus, an information processing method, and a program that can implement a robot system that is accepted by society.

### BACKGROUND ART

In recent years, a system in which a robot provides a service to a person is becoming widespread. In such a system, the robot is required not to give discomfort to the person who receives the service.

On the other hand, Patent Document 1 discloses a mobile body management method that specifies a traveling direction of a target person on the basis of a captured image from a monitoring camera to search for a route on which a mobile body that provides a service to the target person moves while avoiding a range around the target person.

### CITATION LIST

### Patent Document

### Patent Document 1: Japanese Patent Application Laid-Open No. 2020-144591

### SUMMARY OF THE INVENTION

### Problems to be Solved by the Invention

In a case where communication with an unspecified large number of users is assumed, the robot is required to take an action in accordance with the acceptability of the target user to the robot.

The present disclosure has been made in view of such circumstances, and an object of the present disclosure is to be able to implement a robot system that is accepted by society.

### Solutions to Problems

An information processing apparatus of the present disclosure is an information processing apparatus including an action planner that plans an action of a mobile body on the basis of an acceptability level to the mobile body set for each of one or a plurality of users and an action parameter that is set on the basis of an action purpose of the mobile body and determines an action guideline of the mobile body.

In an information processing method of the present disclosure, an information processing apparatus plans an action of a mobile body on the basis of an acceptability level to the mobile body set for each of one or a plurality of users and an action parameter that is set on the basis of an action purpose of the mobile body and determines an action guideline of the mobile body.

A program of the present disclosure causes a computer to execute processing of planning an action of a mobile body on the basis of an acceptability level to the mobile body set for each of one or a plurality of users and an action parameter that is set on the basis of an action purpose of the mobile body and determining an action guideline of the mobile body.

In the present disclosure, an action of a mobile body is planned on the basis of an acceptability level to the mobile body set for each of one or a plurality of users and an action parameter that is set on the basis of an action purpose of the mobile body and determines an action guideline of the mobile body.

### BRIEF DESCRIPTION OF DRAWINGS

FIG. 1 is a diagram illustrating an outline of a robot system to which the technology of the present disclosure can be applied.

FIG. 2 is a block diagram illustrating a hardware configuration example of an information processing apparatus.

FIG. 3 is a block diagram illustrating a functional configuration example of the information processing apparatus.

FIG. 4 is a diagram illustrating an example of an acceptability level to a robot for each user.

FIG. 5 is a flowchart for describing a flow of action plan processing of the robot.

FIG. 6 is a flowchart for describing a specific example of a route calculation.

FIG. 7 is a diagram illustrating an example of a movement route.

FIG. 8 is a diagram illustrating another example of the movement route.

FIG. 9 is a block diagram illustrating a functional configuration example of the information processing apparatus and the robot.

### MODE FOR CARRYING OUT THE INVENTION

Hereinafter, modes for carrying out the present disclosure (hereinafter referred to as embodiments) will be described. Note that the description will be made in the following order.

1. Related art and problems thereof

2. Outline of robot system

3. Configuration of information processing apparatus

4. Flow of action plan processing

5. Specific example of route calculation

6. Modifications

### <1. Related Art and Problems Thereof>

In recent years, in a general environment including a public space such as a road, a station, or an airport, a factory, a warehouse, and the like, robot conveyance by autonomous movement is becoming common due to advancement of sensing technology of robots. Such a system is designed to take a certain distance in consideration of safety when a pedestrian or the like is detected.

For example, a route search approach as proposed in Peter E. Hart; Nils J. Nilsson; Bertram Raphael (July 1968). “A Formal Basis for the Heuristic Determination of Minimum Cost Paths”. IEEE Transactions on Systems Science and Cybernetics 4 (2): 100-107. doi: 10.1109/TSSC.1968.300136. ISSN 0536-1567. is significantly effective in action planning for a transfer robot or the like that achieves a goal by moving.

In addition, in a system in which a robot provides a service to a person, the robot is required not to give discomfort to the person who receives the service.

On the other hand, Patent Document 1 (Japanese Patent Application Laid-Open No. 2020-144591) discloses a mobile body management method that specifies a traveling direction of a target person on the basis of a captured image from a monitoring camera to search for a route on which a mobile body that provides a service to the target person moves while avoiding a range around the target person.

However, for example, it is known that a distance that the robot should take with the pedestrian varies depending on the acceptability and action of the pedestrian to the robot. As a result, it is not always optimal to set a certain distance between the robot and the pedestrian. It is therefore required to propose an action plan that avoids contact with the robot in consideration of a personal space and the traveling direction of the person.

For example, in a domestic communication robot rather than a system for an unspecified large number of users, a mechanism of changing communication in consideration of a degree of intimacy with a family and the like has been proposed, as disclosed in Japanese Patent Application Laid-Open No. 2001-212783. Parameters such as the degree of intimacy assume that a specific user and the robot repeatedly communicate with each other.

On the other hand, in a case where communication with an unspecified large number of users is assumed, the robot is required to take an action in accordance with the acceptability of the target user to the robot. For example, it is assumed that an interaction between the transfer robot and the pedestrian occurs a limited number of times such as one to several times. In such an interaction, it is necessary to maximize social acceptability and movement efficiency by evaluating the degree of intimacy of the user to the robot and maximizing the evaluable parameters.

That is, in a robot that communicates with an unspecified large number of users, it is important to take a behavior and action according to a target user in order to ensure safety and security and smoothly avoid a risk.

Therefore, the technology of the present disclosure makes it possible to implement a robot system that is accepted by society by planning an action of the robot on the basis of an acceptability level of a user to the robot and an action parameter that determines an action guideline of the robot.

### <2. Outline of Robot System>

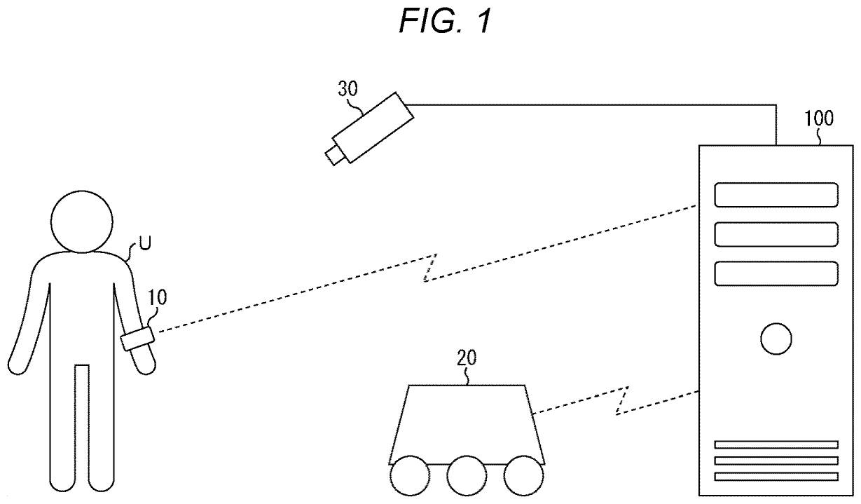

FIG. 1 is a diagram illustrating an outline of a robot system to which the technology of the present disclosure can be applied.

The robot system in FIG. 1 includes a wearable device 10, a robot 20, a sensor group 30, and an information processing apparatus 100.

In the robot system in FIG. 1, communication between an unspecified large number of users U and the robot 20 in a public space is assumed.

The public space referred to herein may be not only a highly public space such as a road, a station, an airport, or a shopping mall, but also a space in which an unspecified large number of users U exist, such as, for example, a warehouse of a company or a house in which a family lives.

The wearable device 10 is worn on a wrist, an arm, a head, or the like of the user U. The wearable device 10 is configured as, for example, a smart watch worn on a wrist, smart glasses worn like glasses, or the like. Furthermore, the wearable device 10 may be configured as a handheld small terminal that can be carried by the user U, such as a smartphone.

The wearable device 10 has a function of performing biological sensing of the user U, and acquires biological information of the user U. The biological information includes at least one of heart rate information, pulse information, perspiration information, respiration information, or exercise information of the user U. The acquired biological information is transmitted to the information processing apparatus 100 via a network such as the Internet by a predetermined communication method including near field communication.

The robot 20 is configured as a mobile body according to the present disclosure. The robot 20 is configured as a robot that autonomously moves from a first point to a second point on the basis of an action plan by the information processing apparatus 100 in a public space where an unspecified large number of users U exist.

Hereinafter, the robot 20 is assumed to be configured as a vehicle-type mobile body that travels on the ground, such as a transfer robot, but may be configured as a human-type or animal-type robot that walks on the ground, a drone that flies in the air, or the like.

The robot 20 is equipped with sensors such as a camera and a distance measuring sensor, and acquires sensor information by sensing a surrounding environment of the robot 20. The robot 20 can search for a route to a destination by simultaneously performing self-localization estimation and environmental map creation by simultaneous localization and mapping (SLAM) by using the acquired sensor information. Furthermore, the acquired sensor information is transmitted to the information processing apparatus 100 via a network such as the

Internet by a predetermined communication method including near field communication as necessary.

Some of the sensors mounted on the robot 20 may be configured to be able to the acquire biological information of the user U acquired by the wearable device 10.

The robot 20 moves in a public space under the control of a company or an individual on the basis of a given action purpose. As the action purpose, the robot 20 is given various tasks and roles by the company or individual who manages the robot 20. The action purpose of the robot 20 includes, for example, a delivery business of picking up and delivering packages, an advertisement business of widely spreading information while moving, and the like.

Note that, in the example in FIG. 1, only one user U is shown in the public space, but it is assumed that a plurality of users U actually exists. In addition, in the public space, the number of robots 20 is not limited to one, and a plurality of robots may exist.

The sensor group 30 includes sensors and devices fixedly installed in a public space where an unspecified large number of users U exist. The sensor group 30 includes, for example, a monitoring camera. The sensor group 30 acquires the sensor information by sensing the public space. The acquired sensor information is transmitted to the information processing apparatus 100 via a network such as the Internet.

Some of the sensors and devices constituting the sensor group 30 may be configured to be able to the acquire biological information of the user U acquired by the wearable device 10.

The information processing apparatus 100 is an information processing apparatus to which the technology of the present disclosure can be applied, and includes, for example, a computer such as a personal computer (PC).

The information processing apparatus 100 is connected to the wearable device 10, the robot 20, and the sensor group 30 in a wired or wireless manner, and executes various types of processing by collecting information from each configuration. In addition, the information processing apparatus 100 transmits execution results of various types of processing to each configuration and controls each configuration as necessary.

<3. Configuration of Information Processing Apparatus>

Here, a detailed configuration of the information processing apparatus 100 will be described.

### (Hardware Configuration Example of Information Processing Apparatus)

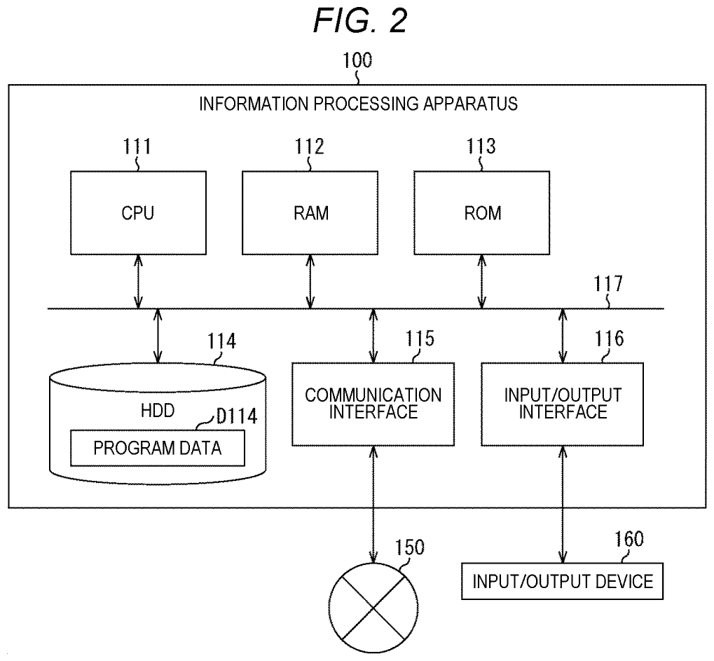

FIG. 2 is a block diagram illustrating a hardware configuration example of the information processing apparatus 100.

The information processing apparatus 100 includes a central processing unit (CPU) 111, a random access memory (RAM) 112, a read only memory (ROM) 113, a hard disk drive (HDD) 114, a communication interface 115, and an input/output interface 116. Each unit of the information processing apparatus 100 is connected by a bus 117.

The CPU 111 operates on the basis of a program stored in the ROM 113 or the HDD 114, and controls each unit. For example, the CPU 111 develops the program stored in the ROM 113 or the HDD 114 in the RAM 112, and executes processing corresponding to various programs.

The ROM 113 stores a boot program such as a basic input output system (BIOS) executed by the CPU 111 when the information processing apparatus 100 is activated, a program depending on hardware of the information processing apparatus 100, and the like.

The HDD 114 is a computer-readable recording medium that non-transiently records the program executed by the CPU 111, data used by the program and the like. Specifically, the HDD 114 is a recording medium that records a program according to the present disclosure, which is an example of program data D114.

The communication interface 115 is an interface for the information processing apparatus 100 to connect to an external network 150 such as the Internet, for example. For example, the CPU 111 receives data from another device or transmits data generated by the CPU 111 to another device via the communication interface 115.

The input/output interface 116 is an interface for connecting an input/output device 160 and the information processing apparatus 100. For example, the CPU 111 receives data from an input device such as a keyboard and a mouse via the input/output interface 116. In addition, the CPU 111 transmits data to an output device such as a display, a speaker, or a printer via the input/output interface 116. Furthermore, the input/output interface 116 may function as a media interface that reads a program or the like recorded on a predetermined recording medium (medium). The medium is, for example, an optical recording medium such as a digital versatile disc (DVD) and a phase change rewritable disk (PD), a magneto-optical recording medium such as a magneto-optica disk (MO), a tape medium, a magnetic recording medium, a semiconductor memory, or the like.

The CPU 111 of the information processing apparatus 100 implements various functions by executing a program loaded on the RAM 112. In addition, the HDD 114 stores programs and various types of data according to the present disclosure. Although the CPU 111 reads the program data D114 from the HDD 114 and executes the program data D114, these programs may be acquired from another apparatus via the external network 150.

In addition, the information processing apparatus 100 may be configured by a plurality of apparatuses performing distributed processing in addition to being configured by a single apparatus. Furthermore, the information processing apparatus 100 may refer to not only the internal data stored inside but also external data held in an external server or the like via the external network 150.

### (Functional Configuration Example of Information Processing Apparatus)

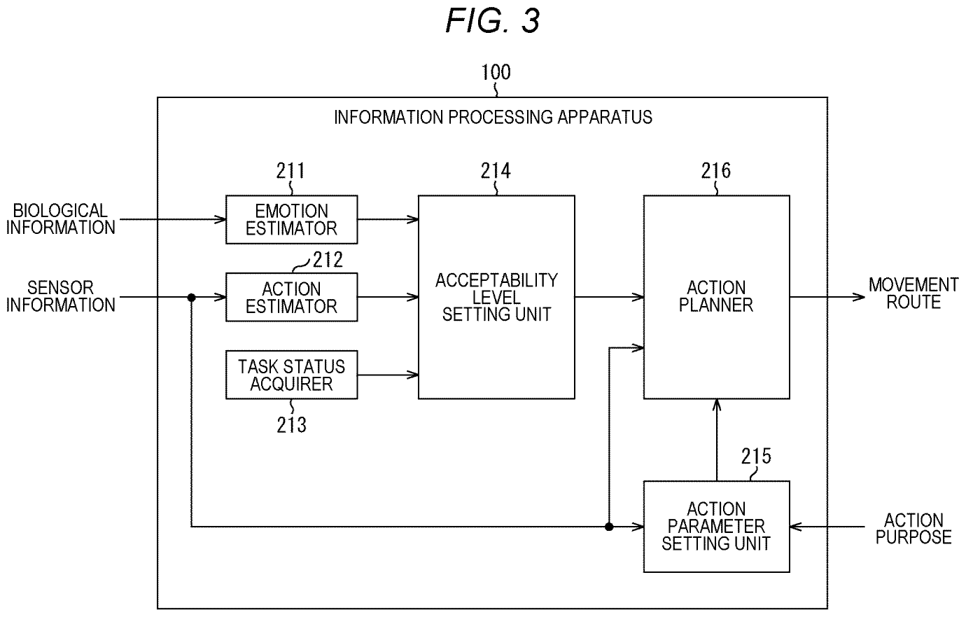

FIG. 3 is a block diagram illustrating a functional configuration example of the information processing apparatus 100.

In the information processing apparatus 100 illustrated in FIG. 3, the CPU 111 executes a predetermined program to implement an emotion estimator 211, an action estimator 212, a task status acquirer 213, an acceptability level setting unit 214, an action parameter setting unit 215, and an action planner 216.

The emotion estimator 211 estimates the emotion of the user toward the robot 20 by using, for example, machine learning on the basis of the biological information such as heart rate information and perspiration information acquired by the wearable device 10 worn by the user. As described above, the biological information may be acquired by the sensors mounted on the robot 20 or the sensor group 30 installed in a public space.

For example, in a case where biological information indicating a relaxed state or a calm state is acquired, the emotion estimator 211 estimates that the user is in a state of being friendly and relieved to the robot 20 as an emotion of the user. On the other hand, in a case where biological information indicating a tense state or an excited state is acquired, the emotion estimator 211 estimates that the user is in a state of being unfriendly and anxious to the robot 20 as an emotion of the user.

Estimated emotion information indicating an emotion of the user is fed to the acceptability level setting unit 214 and stored and accumulated in a storage area (not illustrated). In the storage area (not illustrated), the environment when the emotion of the user is estimated and the type of the target robot 20 may be stored and accumulated together with the emotion information.

The action estimator 212 estimates an action of the user on the basis of the biological information (exercise information) acquired by the wearable device 10 worn by the user, the sensors mounted on the robot 20, and the sensor information acquired by the sensor group 30 installed in a public space. Action information indicating the estimated action of the user is fed to the acceptability level setting unit 214.

For example, the action estimator 212 estimates whether the user is in a moving state or a stationary state on the basis of a captured image from the monitoring camera constituting the sensor group 30. In addition, in a case where the user is in a moving state, the action estimator 212 estimates a moving direction of the user.

Furthermore, the action estimator 212 estimates whether the user is performing an independent action or a group action. In addition, in a case where the user is performing the group action, the action estimator 212 estimates with what member configuration the user is performing the group action.

The task status acquirer 213 acquires a task status of the user. Task information indicating the estimated task status of the user is fed to the acceptability level setting unit 214.

For example, the task status acquirer 213 acquires task information indicating the presence or absence of a task (schedule) of the user from a schedule management application (calendar application) installed in a smartphone or the like owned by the user.

The acceptability level setting unit 214 sets the acceptability level to the robot 20 for each user on the basis of the emotion information from the emotion estimator 211, the action information from the action estimator 212, and the task information from the task status acquirer 213. The acceptability level is an index indicating a degree of acceptability at which the user accepts the presence of the robot 20.

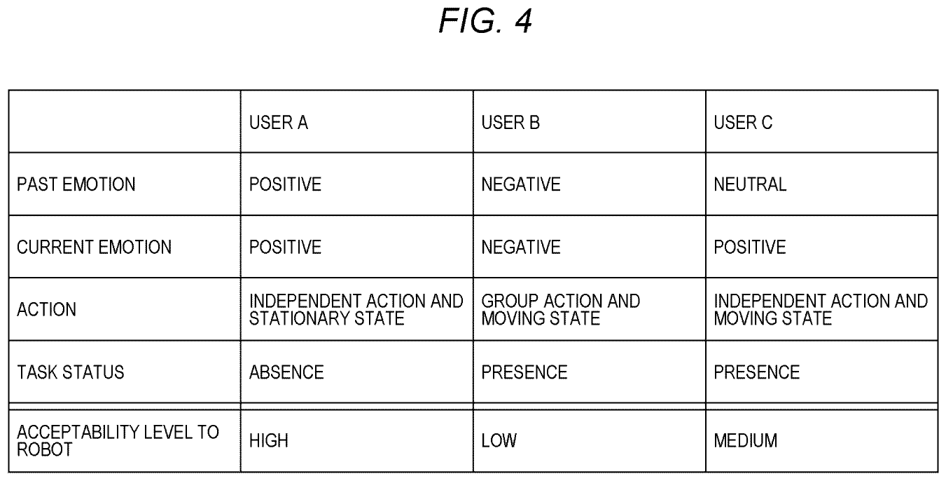

FIG. 4 is a diagram illustrating an example of the acceptability level to the robot for each user at a certain timing.

In the example in FIG. 4, the acceptability level set on the basis of a past emotion, current emotion, action, and task status of each of three users A, B, and C is illustrated.

The past emotion of the user A to the robot is positive, and the current emotion is also positive. In addition, the user A is performing an independent action and is in a stationary state. Furthermore, the user A does not have a task at present. In this case, since the user A has been friendly to the robot both in the past and at present and is in a calm state, the acceptability to the robot is estimated to be high, and the acceptability level of the user A is set to “high”.

The past emotion of the user B to the robot is negative, and the current emotion is also negative. In addition, the user B is performing a group action and is in a moving state. Furthermore, the user B has a task at present. In this case, since the user B is friendly to the robot neither in the past nor at present and is moving with a plurality of people for a certain schedule, the acceptability to the robot is estimated to be low, and the acceptability level of the user B is set to “low”.

In particular, in a case where the user B is accompanied by a child as a member configuration of the group action, when the robot passes near the user B, a psychological load on the child is estimated to be high and the acceptability to the robot is estimated to be low.

The past emotion of the user C to the robot is neutral, and the current emotion is also positive. In addition, the user C is performing an independent action and is in a moving state. Furthermore, the user C has a task at present. In this case, although the user C is friendly to the robot at present, since the user C is moving alone for a certain schedule, the acceptability to the robot is estimated to be moderate, and the acceptability level of the user C is set to “medium”.

As described above, the acceptability level of each user to the robot is set on the basis of the emotion information, the action information, and the task information of each user. The set acceptability level of each user is fed to the action planner 216.

Note that, in the example in FIG. 4, the acceptability level of the user is indicated by three levels of “high”, “medium”, or “low”, but may be indicated by a score value or may be indicated by a binary value based on a magnitude relationship of the score value with respect to a threshold value.

Furthermore, the acceptability level of the user in only required to be set, for example, when the robot 20 starts an action (movement) according to the action purpose, but may be updated in real time or may be updated at predetermined regular time intervals during the movement of the robot 20.

The action parameter setting unit 215 sets an action parameter for determining an action guideline of the robot 20 on the basis of the action purpose of the robot 20, and feeds the action parameter to the action planner 216. As described above, the action purpose of the robot 20 is set in advance by a company or an individual who manages the robot 20, and can be input to the information processing apparatus 100.

The action planner 216 plans an action of the robot 20 on the basis of the acceptability level of each user set by the acceptability level setting unit 214 and the action guideline of the robot 20 determined by the action parameter set by the action parameter setting unit 215. Specifically, the action planner 216 calculates a movement route of the robot 20 on the basis of the acceptability level of each user and the action guideline of the robot 20 determined by the action parameter, and outputs the movement route to the robot 20.

### <4. Flow of Action Plan Processing>

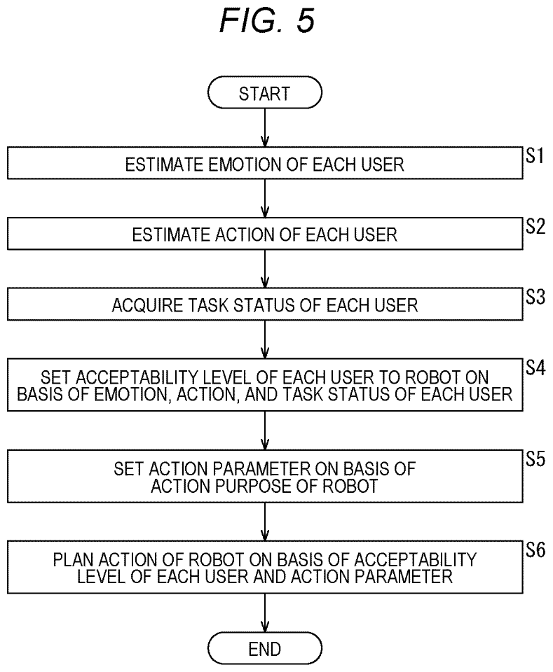

Next, a flow of action plan processing of the robot 20 by the information processing apparatus 100 will be described with reference to a flowchart in FIG. 5. The processing of FIG. 5 is executed, for example, before the robot 20 starts an action (movement) according to the action purpose.

In step S1, the emotion estimator 211 estimates the emotion of each user to the robot 20 on the basis of the biological information acquired by the wearable device 10 worn by each user in the public space.

In step S2, the action estimator 212 estimates an action of the user on the basis of the biological information acquired by the wearable device 10 worn by each user, the sensors mounted on the robot 20, and the sensor information acquired by the sensor group 30 installed in a public space.

In step S3, the task status acquirer 213 acquires a task status of each user.

In step S4, the acceptability level setting unit 214 sets the acceptability level of each user to the robot 20 on the basis of the emotion of each user estimated by the emotion estimator 211, the action of each user estimated by the action estimator 212, and the task status of each user acquired by the task status acquirer 213.

In step S5, the action parameter setting unit 215 sets the action parameter for determining the action guideline of the robot 20 on the basis of the action purpose of the robot 20.

Here, as the action guidelines of the robot 20, two action guidelines of a first action guideline and a second action guideline are defined in accordance with the action purpose of the robot. In this case, the action parameter setting unit 215 sets an emphasis ratio between the first action guideline and the second action guideline as the action parameter.

In step S6, the action planner 216 plans an action of the robot 20 on the basis of the acceptability level of each user set by the acceptability level setting unit 214 and the action guideline of the robot 20 determined by the action parameter set by the action parameter setting unit 215.

Specifically, the action planner 216 calculates the movement route of the robot 20 reflecting the positional relationship with each user based on the acceptability level in accordance with the emphasis ratio between the first action guideline and the second action guideline set as the action parameter.

Here, the first action guideline is defined as an action guideline that emphasizes an action efficiency of the robot 20, and the second action guideline is defined as an action guideline that emphasizes a harmony with the surrounding environment of the robot 20.

Here, the action efficiency of the robot 20 is the movement efficiency or a task execution efficiency for achieving the action purpose, and the harmony with the surrounding environment of the robot 20 represents that a psychological load on the nearby users is minimized.

Conventionally, in an action plan (route search) of a robot, action efficiency has been emphasized. This arrangement is achieved, for example, by calculating the shortest route between the points using an A* (A-star) search algorithm or the like. This configuration is a significantly important action guideline from the viewpoint of workability of executing more tasks.

On the other hand, in a public space, under a situation where it is difficult to solve a problem by the robot alone, it is considered to be necessary to provide assistance from the user and ensure cooperativity with society. That is, as the action guideline, the second action guideline is required, which places an emphasis on harmony with the surrounding environment of the robot 20.

Therefore, the action planner 216 calculates a movement route closer to the shortest route as the emphasis ratio of the first action guideline is higher, regardless of the positional relationship with the user. In addition, the action planner 216 calculates a movement route passing through a position closer to a user whose acceptability level is higher and farther from a user whose acceptability level is lower as the emphasis ratio of the second action guideline is higher, regardless of a movement distance and safety. It is therefore possible to achieve both the action efficiency of the robot 20 and the harmony with the surrounding environment.

### <5. Specific Example of Route Calculation>

Hereinafter, a specific example of the calculation (route calculation) of the movement route of the robot 20 will be described.

The action parameter, that is, the emphasis ratio between the first action guideline and the second action guideline set on the basis of the action purpose of the robot 20 is defined in advance for the action purpose.

For example, in a case where the action purpose of the robot 20 is a home delivery business and the name of a company or the like that manages the robot 20 is not known to surrounding users or the like, the emphasis ratio between the first action guideline (action efficiency) and the second action guideline (harmony with the surrounding environment) is set to 10:0 (emphasis of 100% on the action efficiency).

On the other hand, in a case where the action purpose of the robot 20 is a home delivery business and the name of a company or the like that manages the robot 20 is known to surrounding users or the like, the emphasis ratio between the first action guideline (action efficiency) and the second action guideline (harmony with the surrounding environment) is set to 5:5 (emphasis of 50% on the action efficiency and emphasis of 50% on the harmony with the surrounding environment).

This arrangement is set because, in the home delivery business, in a situation where the name of the company is not known, the action efficiency can be maximally emphasized, whereas in a situation where the name of the company is known, in order to avoid damage to the brand image of the company, harmony with the surrounding environment needs to be emphasized.

In addition, in a case where the action purpose of the robot 20 is an advertisement business, the emphasis ratio between the first action guideline (action efficiency) and the second action guideline (harmony with the surrounding environment) is set to 1:9 (emphasis of 10% on the action efficiency and emphasis of 90% on the harmony with the surrounding environment).

This arrangement is set because, in the advertisement business and the like, in a case where an interaction with a user occurs, for example, in a case where information is widely known to an unspecified large number of users, it is necessary to particularly emphasize harmony with the surrounding environment in order to enhance the acceptability of the user.

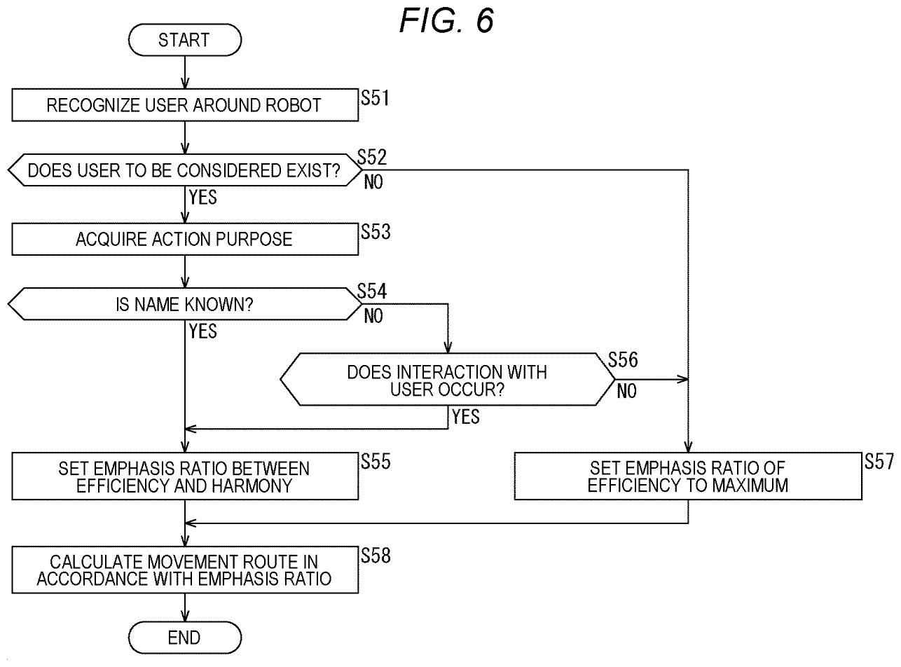

Here, a specific example of the route calculation according to an importance ratio defined in advance for the action purpose as described above will be described with reference to the flowchart in FIG. 6. The processing of FIG. 6 corresponds to steps S5 and S6 of the action plan processing described with reference to FIG. 5.

In step S51, the action parameter setting unit 215 recognizes the user around the robot 20 on the basis of the sensor information acquired by the sensors mounted on the robot 20 or the sensor group 30 installed in the public space.

In step S52, the action parameter setting unit 215 determines whether or not a user to be considered exists on the basis of a recognition result. The user to be considered here is, for example, a user who is within a predetermined range including a route connecting the first point to the second point where the robot 20 moves and has the acceptability level being set.

In a case where it is determined in step S52 that a user to be considered exists, the processing proceeds to step S53, and the action parameter setting unit 215 acquires an action purpose of the robot 20.

In step S54, the action parameter setting unit 215 determines whether or not the name of the company that manages the robot 20 is known to surrounding users or the like from the acquired action purpose.

In a case where it is determined in step S54 that the name is known, the processing proceeds to step S55, and the action parameter setting unit 215 sets an importance ratio between the action efficiency (first action guideline) and the harmony with the surrounding environment (second action guideline). Here, the emphasis ratio between the action efficiency and the harmony with the surrounding environment is set to 5:5 (emphasis of 50% on the action efficiency and emphasis of 50% on the harmony with the surrounding environment).

On the other hand, in a case where it is determined in step S54 that the name is known, the processing proceeds to step S56, and the action parameter setting unit 215 determines whether or not an interaction with the user occurs for the acquired action purpose.

In a case where it is determined in step S56 that an interaction with the user occurs, the processing proceeds to step S55, and the action parameter setting unit 215 sets an importance ratio between the action efficiency (first action guideline) and the harmony with the surrounding environment (second action guideline). Here, the emphasis ratio between the action efficiency and the harmony with the surrounding environment is set to 1:9 (emphasis of 10% on the action efficiency and emphasis of 90% on the harmony with the surrounding environment).

On the other hand, in a case where it is determined in step S52 that no user to be considered exists, or in a case where it is determined in step S56 that no interaction with the user occurs, the processing proceeds to step S57.

In step S57, the action parameter setting unit 215 sets the importance ratio of the action efficiency (first action guideline) to the maximum (emphasis of 100% on the action efficiency).

After step S55 or step S57, the processing proceeds to step S58, and the action planner 216 calculates the movement route of the robot 20 in accordance with the set emphasis ratio. At this time, the movement route of the robot 20 reflecting the positional relationship with each user is calculated on the basis of the acceptability level of the user to be considered existing around the robot 20.

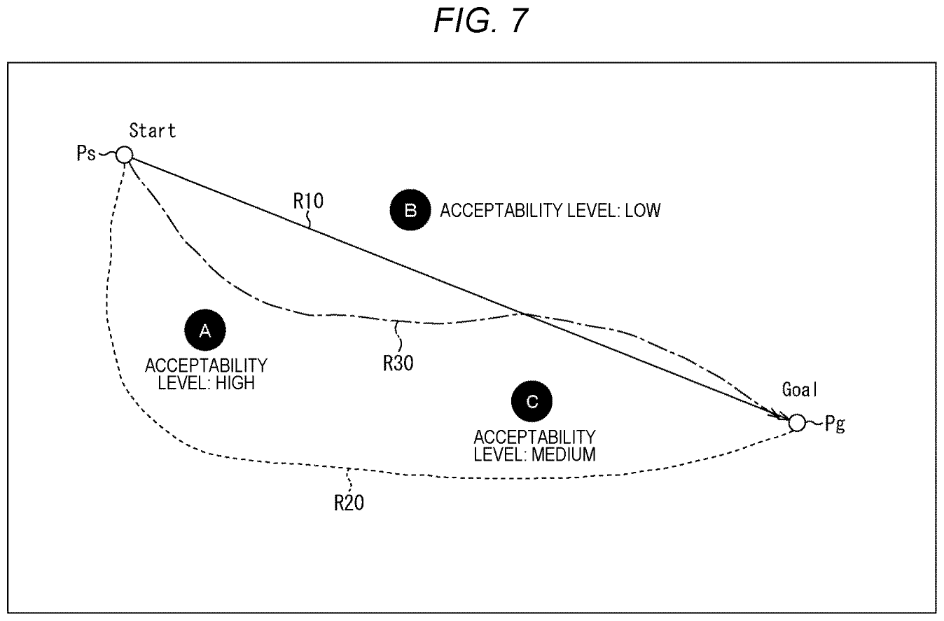

For example, as illustrated in FIG. 7, it is assumed that the robot 20 moves from a start point Ps to a goal point Pg. In a range including the route on which the robot 20 moves, three users A, B, and C who have the acceptability level described with reference to FIG. 4 being set exist.

In such a state, in a case where the importance ratio of the action efficiency (first action guideline) is set to the maximum (emphasis of 100% on the action efficiency), a route R10 indicated by a solid line is calculated as the movement route of the robot 20. In the example in FIG. 7, the route R10 is a straight line that is the shortest route from the start point Ps to the goal point Pg, but actually, in order to avoid contact with the users A, B, and C, the route R is a route on which the robot 20 moves at the shortest distance while keeping a certain distance from each user.

On the other hand, in a case where the importance ratio of the harmony with the surrounding environment (second action guideline) is set to the maximum (emphasis of 100% on the harmony with the surrounding environment), a route R20 indicated by a broken line is calculated as the movement route of the robot 20. It is often accepted to approach a user who is friendly to the robot 20, while it is often not accepted to approach a user who is not friendly to the robot 20. Therefore, in the example in FIG. 7, the route R20 is a route passing through a position close to the user A with higher acceptability level and farther from the user B with lower acceptability level.

In a case where the emphasis ratio between the action efficiency and the surrounding environment is set to a certain ratio, a route R30 corresponding to each ratio of the action efficiency and the surrounding environment is calculated. For example, a route that is an arithmetic average of the route R10 in a case where emphasis of 100% on the action efficiency is set and the route R20 in a case where emphasis of 100% on the harmony with the surrounding environment is set is calculated in accordance with the emphasis ratio. In the example of FIG. 7, the route R30 indicated by a one-dot chain line is a route in a case where emphasis of 50% on the action efficiency and emphasis of 50% on the harmony with the surrounding environment are set.

As described above, the action of the robot 20 is planned on the basis of the acceptability level to the robot 20 set for each user and the action parameter that determines the action guideline of the robot 20. As a result, the robot 20 can take an action according to the acceptability of the user to the robot 20, and it is possible to implement a robot system that is accepted by the society.

Specifically, since the movement route of the robot 20 reflecting the positional relationship with each user based on the acceptability level is calculated in accordance with the emphasis ratio between the first action guideline and the second action guideline, it is possible to achieve both the action efficiency and the harmony with the surrounding environment of the robot 20.

As a use case of the route calculation as described above, there can be a corporate activity or advertisement targeting a specific user group.

In the corporate activities and advertisement, it is important to enhance affinity of the user group and increase engagement. For this purpose, it is required to specify a target user group and perform behavior that the users feel friendly.

Therefore, when the robot 20 passes by each user who can be a target, it is possible to increase engagement and implement corporate activities and advertisement promotion that are accepted by society by preferentially passing around the user with high affinity.

### <6. Modifications>

Hereinafter, modifications of the above embodiments will be described.

### (Route Calculation According to Number of Users)

In the example in FIG. 7, in a case where the emphasis ratio is set to a certain ratio, the movement route of the robot 20 reflecting only the positional relationship with the user having a higher acceptability level than a certain degree may be calculated in accordance with the number of users to be considered (users existing around the robot 20). This calculation is implemented by the action planner 216 recognizing the number of users existing around the robot 20 on the basis of, for example, the sensor information from the sensors mounted on the robot 20 and the sensor group 30.

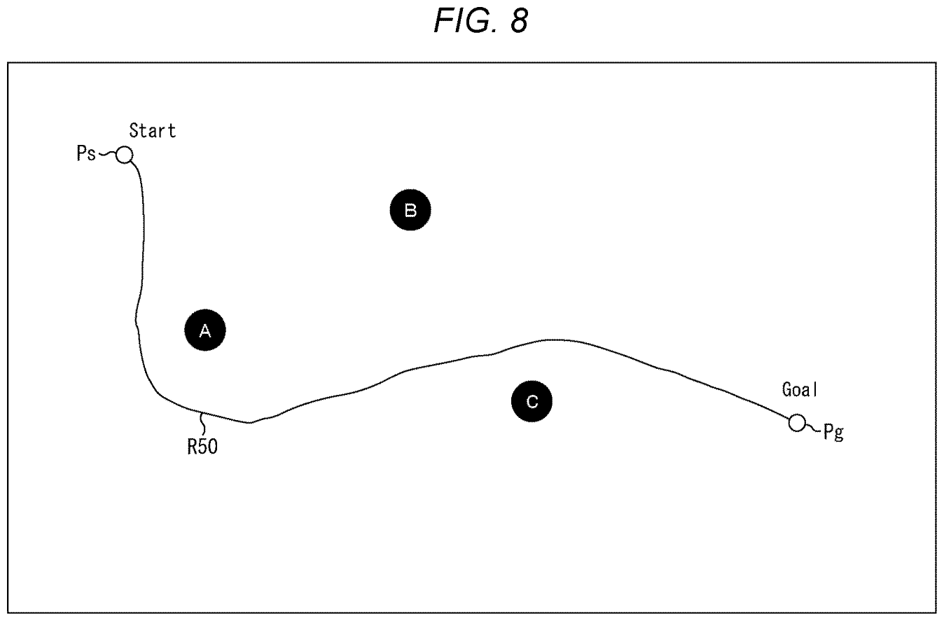

For example, in a case where emphasis of 50% on the action efficiency and emphasis of 50% on the harmony with the surrounding environment are set, as illustrated in FIG. 8, a route R50 reflecting only the positional relationship with the user A having high acceptability level (considering only passing near the user A) may be calculated.

### (Setting of Emphasis Ratio)

The emphasis ratio between the action efficiency and the surrounding environment may change in accordance with the number of users to be considered (users existing around the robot 20). This calculation is implemented by the action parameter setting unit 215 recognizing the number of users existing around the robot 20 on the basis of, for example, the sensor information from the sensors mounted on the robot 20 and the sensor group 30.

For example, in a case where there is no user to be considered around the robot 20 while the robot 20 is moving, even if the importance ratio of the harmony with the surrounding environment has been set to be higher, the importance ratio may be changed and the importance ratio of the action efficiency may be set to be higher.

In addition, a predetermined (defined) emphasis ratio may be set in a specific area as the emphasis ratio of between the action efficiency and the harmony with the surrounding environment. This calculation is implemented by the action parameter setting unit 215 recognizing the position of the robot 20 on the basis of, for example, the sensor information from the sensors mounted on the robot 20.

For example, in a case where the robot 20 moves in an urban area, regardless of the presence or absence of a user around the robot 20, the emphasis ratio on the harmony with the surrounding environment is set to be higher. In addition, in a place with a large traffic volume, the emphasis ratio of the action efficiency is set to be higher in consideration of safety.

### (Robot Including Action Planner)

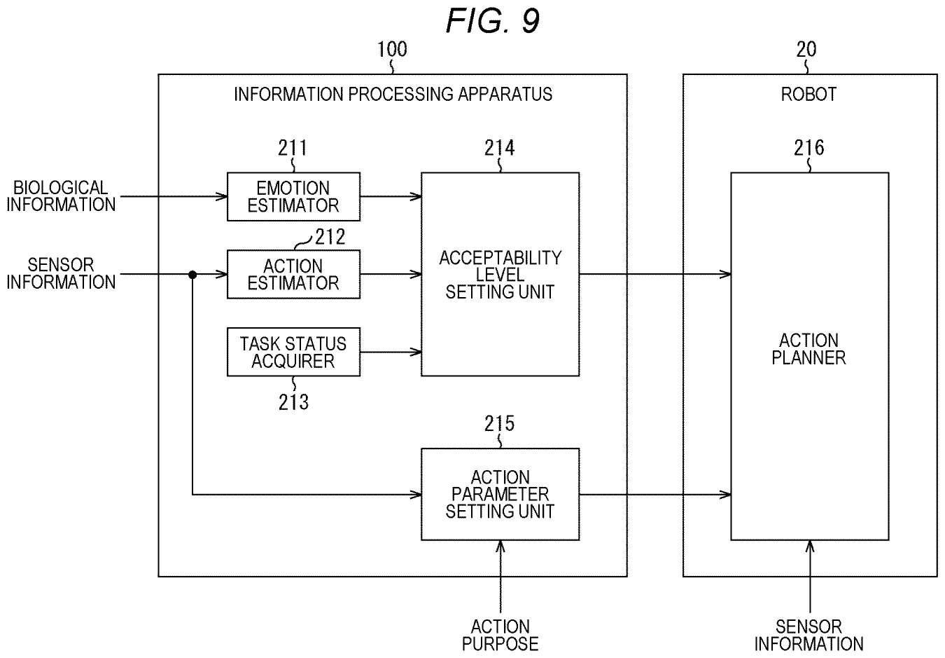

As illustrated in FIG. 9, by including the action planner 216, the robot 20 may be configured as an information processing apparatus to which the technology of the present disclosure can be applied. In this case, the robot 20 can plan its own action on the basis of the acceptability level of each user set by the information processing apparatus 100 and the action guideline of the robot 20 determined by the action parameter.

Note that, in the above configuration, the movement route reflecting the positional relationship with each user based on the acceptability level is calculated by the action planner 216. However, in an environment where a plurality of robots 20 exist, a movement route reflecting the positional relationship between the robots 20 may be calculated.

The embodiments of the technology of the present disclosure are not limited to the embodiments described above, and various modifications may be made without departing from the scope of the technology of the present disclosure.

For example, the technology of the present disclosure can have a configuration of cloud computing in which one function is shared and processed in cooperation by a plurality of devices via a network.

In addition, each step described in the flowcharts can be executed by one apparatus, or can be executed by being shared by a plurality of apparatuses.

Furthermore, in a case where a plurality of pieces of processing is included in one step, the plurality of pieces of processing included in the one step can be executed by being shared by a plurality of apparatuses, in addition to being executed by one apparatus.

The effects described in the present specification are merely examples and are not limited, and other effects may be provided.

Furthermore, the technology of the present disclosure can have the following configurations.

(1)

An information processing apparatus including an action planner that plans an action of a mobile body on the basis of an acceptability level to the mobile body set for each of one or a plurality of users and an action parameter that is set on the basis of an action purpose of the mobile body and determines an action guideline of the mobile body.

(2)

The information processing apparatus according to (1), in which

the acceptability level is set on the basis of at least an emotion of the user to the mobile body.

(3)

The information processing apparatus according to (2), in which

the emotion of the user is estimated on the basis of biological information of the user.

(4)

The information processing apparatus according to (3), in which

the biological information includes at least one of heart rate information, pulse information, perspiration information, respiration information, or exercise information.

(5)

The information processing apparatus according to (4), in which

the biological information is acquired by at least any of a wearable device worn by the user, sensors mounted on the mobile body, or a sensor group provided in a space in which the user exists.

(6)

The information processing apparatus according to (5), in which

the acceptability level is set on the basis of the emotion of the user, an action of the user, and a task status of the user, and

the action of the user is estimated on the basis of another sensor information acquired by at least any of the wearable device, the sensors mounted on the mobile body, or the sensor group.

(7)

The information processing apparatus according to any of (1) to (6), in which

an emphasis ratio between a first action guideline and a second action guideline is set as the action parameter.

(8)

The information processing apparatus according to (7), in which

the first action guideline is an action guideline that emphasizes an action efficiency of the mobile body, and

the second action guideline is an action guideline that emphasizes harmony with a surrounding environment of the mobile body.

(9)

The information processing apparatus according to (8), in which

the action planner calculates a movement route of the mobile body reflecting a positional relationship with the user based on the acceptability level in accordance with the emphasis ratio.

(10)

The information processing apparatus according to (9), in which

the action planner calculates the movement route passing through a position closer to the user whose acceptability level is higher and farther from the user whose acceptability level is lower as the emphasis ratio of the second action guideline is higher.

(11)

The information processing apparatus according to (9) or (10), in which

the action planner calculates the movement route reflecting only a positional relationship with the user having the acceptability level higher than a certain degree in accordance with the number of users existing around the mobile body.

(12)

The information processing apparatus according to any of (8) to (11), in which

the emphasis ratio changes in accordance with the number of users existing around the mobile body.

(13)

The information processing apparatus according to any of (8) to (11), in which

the emphasis ratio predetermined is set in a specific area.

(14)

The information processing apparatus according to any of (8) to (11), in which

the emphasis ratio of the second action guideline is set higher in a case where a specific name related to the action purpose is known to the user during movement of the mobile body.

(15)

The information processing apparatus according to any of (8) to (11), in which

the emphasis ratio of the second action guideline is set higher in a case where the action purpose includes an interaction with the user.

(16)

The information processing apparatus according to any of (1) to (15), in which

the mobile body includes a robot that autonomously moves from a first point to a second point.

(17)

An information processing method, in which

an information processing apparatus

plans an action of a mobile body on the basis of an acceptability level to the mobile body set for each of one or a plurality of users and an action parameter that is set on the basis of an action purpose of the mobile body and determines an action guideline of the mobile body.

(18)

A program causing a computer to execute processing of

planning an action of a mobile body on the basis of an acceptability level to the mobile body set for each of one or a plurality of users and an action parameter that is set on the basis of an action purpose of the mobile body and determining an action guideline of the mobile body.

### REFERENCE SIGNS LIST

10 Wearable device

### 20 Robot

30 Sensor group
100 Information processing apparatus

### 111 CPU

211 Emotion estimator
212 Action estimator
213 Task status acquirer
214 Acceptability level setting unit
215 Action parameter setting unit
216 Action planner

1. An information processing apparatus comprising:
an action planner that plans an action of a mobile body on a basis of an acceptability level to the mobile body set for each of one or a plurality of users and an action parameter that is set on a basis of an action purpose of the mobile body and determines an action guideline of the mobile body.

2. The information processing apparatus according to claim 1, wherein
the acceptability level is set on a basis of at least an emotion of the user to the mobile body.

3. The information processing apparatus according to claim 2, wherein
the emotion of the user is estimated on a basis of biological information of the user.

4. The information processing apparatus according to claim 3, wherein
the biological information includes at least one of heart rate information, pulse information, perspiration information, respiration information, or exercise information.

5. The information processing apparatus according to claim 4, wherein
the biological information is acquired by at least any of a wearable device worn by the user, sensors mounted on the mobile body, or a sensor group provided in a space in which the user exists.

6. The information processing apparatus according to claim 5, wherein
the acceptability level is set on a basis of the emotion of the user, an action of the user, and a task status of the user, and
the action of the user is estimated on a basis of another sensor information acquired by at least any of the wearable device, the sensors mounted on the mobile body, or the sensor group.

7. The information processing apparatus according to claim 1, wherein
an emphasis ratio between a first action guideline and a second action guideline is set as the action parameter.

8. The information processing apparatus according to claim 7, wherein
the first action guideline is an action guideline that emphasizes an action efficiency of the mobile body, and
the second action guideline is an action guideline that emphasizes harmony with a surrounding environment of the mobile body.

9. The information processing apparatus according to claim 8, wherein
the action planner calculates a movement route of the mobile body reflecting a positional relationship with the user based on the acceptability level in accordance with the emphasis ratio.

10. The information processing apparatus according to claim 9, wherein
the action planner calculates the movement route passing through a position closer to the user whose acceptability level is higher and farther from the user whose acceptability level is lower as the emphasis ratio of the second action guideline is higher.

11. The information processing apparatus according to claim 9, wherein
the action planner calculates the movement route reflecting only a positional relationship with the user having the acceptability level higher than a certain degree in accordance with a number of users existing around the mobile body.

12. The information processing apparatus according to claim 8, wherein
the emphasis ratio changes in accordance with the number of users existing around the mobile body.

13. The information processing apparatus according to claim 8, wherein
the emphasis ratio predetermined is set in a specific area.

14. The information processing apparatus according to claim 8, wherein
the emphasis ratio of the second action guideline is set higher in a case where a specific name related to the action purpose is known to the user during movement of the mobile body.

15. The information processing apparatus according to claim 8, wherein
the emphasis ratio of the second action guideline is set higher in a case where the action purpose includes an interaction with the user.

16. The information processing apparatus according to claim 1, wherein
the mobile body includes a robot that autonomously moves from a first point to a second point.

17. An information processing method, wherein
an information processing apparatus
plans an action of a mobile body on a basis of an acceptability level to the mobile body set for each of one or a plurality of users and an action parameter that is set on a basis of an action purpose of the mobile body and determines an action guideline of the mobile body.

18. A program that causes a computer to execute processing of
planning an action of a mobile body on a basis of an acceptability level to the mobile body set for each of one or a plurality of users and an action parameter that is set on a basis of an action purpose of the mobile body and determining an action guideline of the mobile body.
